SEQUENCE LISTING (28 sequences)

SEQ ID NO: 1 - protein, 10 aa, Human immunodeficiency virus
RGPGRAFVTI

SEQ ID NO: 2 - protein, 9 aa, Human immunodeficiency virus
AMQMLKETI

SEQ ID NO: 3 - protein, 5 aa, Human immunodeficiency virus
SLVEL

SEQ ID NO: 4 - protein, 5 aa, Human immunodeficiency virus
SLVEL

SEQ ID NO: 5 - protein, 6 aa, Human immunodeficiency virus
SGPVEL

SEQ ID NO: 6 - protein, 6 aa, Human immunodeficiency virus
SGPVEL

SEQ ID NO: 7 - protein, 4 aa, Human immunodeficiency virus
LVEL

SEQ ID NO: 8 - protein, 4 aa, Human immunodeficiency virus
LVEL

SEQ ID NO: 9 - protein, 5 aa, Human immunodeficiency virus
GPVEL

SEQ ID NO: 10 - protein, 5 aa, Human immunodeficiency virus
GPVEL

SEQ ID NO: 11 - DNA, 69 bp, Human immunodeficiency virus
gaggtgcagctgttggagtctgggggaggcttggtacagcctggggggtccctgagactctcctgtgca

SEQ ID NO: 12 - DNA, 78 bp, Human immunodeficiency virus
ccctggagcctggcggacccagctcatggcatagctgctaaaggtgaatccagaggctgcacaggagagtctcaggga

SEQ ID NO: 13 - DNA, 42 bp, Human immunodeficiency virus
tgggtccgccaggctccagggaaggggctggagtgggtctca

SEQ ID NO: 14 - DNA, 78 bp, Human immunodeficiency virus
gaaccggcccttcacggagtctgcgtagtatgtgctaccaccactaccactaatagctgagacccactccagcccctt

SEQ ID NO: 15 - DNA, 69 bp, Human immunodeficiency virus
gactccgtgaagggccggttcaccatctccagagacaattccaagaacacgctgtatctgcaaatgaac

SEQ ID NO: 16 - DNA, 60 bp, Human immunodeficiency virus
cgcacagtaatatacggccgtgtcctcggctctcaggctgttcatttgcagatacagcgt

SEQ ID NO: 17 - DNA, 60 bp, Human immunodeficiency virus
gccgtatattactgtgcgaaaggtagttactttgactactggggccagggaaccctggtc

SEQ ID NO: 18 - DNA, 33 bp, Human immunodeficiency virus
tgaggagacggtgaccagggttccctggcccca

SEQ ID NO: 19 - DNA, 47 bp, Human immunodeficiency virus
  primer (1)..(47)
attctagccatggtgaattcctgcaggaggtgcagctgttggagtct

SEQ ID NO: 20 - DNA, 40 bp, Human immunodeficiency virus
  primer (1)..(40)
catgtacgtatggatccattgaggagacggtgaccagggt

SEQ ID NO: 21 - DNA, 66 bp, Human immunodeficiency virus
gccgtatattactgtgcgcgtggtcctggtcgtgcttttgttactatttggggccagggaaccctg

SEQ ID NO: 22 - DNA, 60 bp, Human immunodeficiency virus
  misc_feature (16)..(17): n is a, c, g, or t
gtatattactgtgcgnnkggtnnkggtnnkgctnnkgttnnkatttggggccagggaacc

SEQ ID NO: 23 - DNA, 60 bp, Human immunodeficiency virus
  misc_feature (28)..(29): n is a, c, g, or t
gtatattactgtgcgcgtggtcctggtnnkgctnnknnknnknnktggggccagggaacc

SEQ ID NO: 24 - DNA, 39 bp, Human immunodeficiency virus
  primer (1)..(39)
tgatattcgtactcgagccatggtgtatattactgtgcg

SEQ ID NO: 25 - DNA, 40 bp, Human immunodeficiency virus
  primer (1)..(40)
atgattgacaaagcttggatccctaggttccctggcccca

SEQ ID NO: 26 - DNA, 69 bp, Human immunodeficiency virus
gtatattactgtgcgcaggctgctatgcagatgcttaaggagactattaacgagtggggccagggaacc

SEQ ID NO: 27 - DNA, 69 bp, Human immunodeficiency virus
  misc_feature (28)..(29): n is a, c, g, or t
gtatattactgtgcgcaggctgctatgnnkatgcttnnknnknnkattaacgagtggggccagggaacc

SEQ ID NO: 28 - DNA, 69 bp, Human immunodeficiency virus
  misc_feature (25)..(26): n is a, c, g, or t
gtatattactgtgcgcaggctgctnnkcagnnkcttnnkgagactnnkaacgagtggggccagggaacc

(19) United States
(12) Patent Application Publication
(10) Pub. No.: US 2009/0214591 A1
(43) Pub. Date: Aug. 27, 2009

(54) Immunogens for Vaccines Against Antigenically Variable Pathogens and Diseases

(75) Inventors: Karen Manucharyan, Mexico (MX); Gohar Gevorgyan, Mexico (MX)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: PRIMEX CLINICAL LABORATORIES, INC., Van Nuys, CA (US)

(21) Appl. No.: 11/908,942

(22) PCT Filed: Mar. 17, 2006
(86) PCT No.: PCT/US2006/009751

§ 371 Date: Feb. 12, 2009

Related U.S. Application Data
(60) Provisional application No. 60/662,748, filed on Mar. 17, 2005.

(51) Int. Cl. A61K 39/21; A61K 39/00; A61K 39/12; A61P 37/04

(52) U.S. Cl. 424/208.1; 424/184.1; 424/204.1

(57) ABSTRACT

The present invention provides compositions and methods for the therapeutic and/or prophylactic treatment of pathogen infections and/or disease states. The compositions may comprise variable epitope libraries (VELs), containing antigenic epitopes with one or more amino acid substitutions in the native epitope sequence. In preferred embodiments, the substituted amino acid may be replaced with each of the 19 other naturally occurring amino acids. In more preferred embodiments, multiple amino acid residues may be substituted. Such compositions and methods may be of use for production of vaccines against pathogens or diseases that show a high degree of genetic variability.

### TECHNICAL FIELD

The present invention relates to methods and compositions of immunogens for vaccines or treatment directed against antigenically variable regions of pathogens and diseases.

### BACKGROUND ART

Recognition of one macromolecule by another is a key event and the specificity of this interaction is its most important aspect. In the search for novel targets and identifying molecules, researchers looked to complement existing natural compounds which have been extensively screened, with a novel and diversified group of molecules not found in nature. As such, combinatorial libraries of synthesized novel compounds including nucleic or amino acid sequences may be synthesized for targeting identifying antigens for directing treatments to cells, diagnosing conditions and drug development.

One obstacle in the advancement for developing vaccines against pathogens with genetic variability is immune escape. This is characterized by amino acid substitutions in specific regions (epitopes) of pathogen's antigens recognized by the host immune system (CTL, Th and B epitopes). Despite the degenerate nature of the interactions between a TCR of T cells and MHC/peptide complex on antigen-presenting cells, the majority of circulation variants are not recognized by CTLs as seen with HIV (Human Immunodeficiency Virus) and SIV (Simian Immunodeficiency Virus) infections. This may explain the immune system's failure in clearing or containing these viruses. But it is also an indication that there is a little chance that the reported HIV/AIDS vaccines currently undergoing animal/clinical testing will be effective. The immune escape caused by mutations in epitopes or flanking regions (affecting the correct epitope processing) is an ongoing dynamic process. The end result is complex interactions between viral fitness cost of mutations, immune pressure exerted by the host, host genetic factors and viral load.

Because of the dynamic and elusive nature of these pathogens, a new vaccine concept based on application of variable epitope libraries (VELs) is needed to target variable pathogens, such as HIV, SIV, HCV, influenza and some cancers.

### DISCLOSURE OF THE INVENTION

The present invention provides for VELs compositions and methods of use for treatment of disease. In one embodiment of the present invention, a composition may include a synthetic peptide. In accordance with this embodiment, the synthetic peptide may include at least one epitope of a pathogen- or disease-specific polypeptide, where at least one amino acid residue of the peptide is substituted with each of the other nineteen common amino acid residues.

In another embodiment, a composition may include a synthetic peptide with at least one epitope of a pathogen- or disease-specific polypeptide where every other amino acid residue of the peptide is substituted with one of the other nineteen common amino acid residues such as every even amino acid residue of the peptide or every odd amino acid residue of the peptide.

In one example, the composition of the synthetic peptide disclosed herein may be prepared by expression in a bacterial, viral or eukaryotic expression system. In another example, the composition of the peptide may be expressed and displayed on the surface of a recombinant bacteriophage, bacterium or yeast cell. In accordance with these embodiments, the composition of an epitope of a pathogen-specific polypeptide disclosed herein may be selected from one or more epitopes of a Human Immunodeficiency Virus (HIV)-specific polypeptide, a Simian Immunodeficiency Virus (SIV)-specific polypeptide, a Hepatitis A-specific polypeptide, a Hepatitis B-specific polypeptide, a Hepatitis C-specific polypeptide, a rhinovirus-specific polypeptide, an influenza virus-specific polypeptide, and a plasmodium falciparum-specific polypeptide. Alternatively, the epitope of a disease-specific polypeptide may be one or more epitopes of a tumor associated antigen (TAA).

In another embodiment of the present invention, a method for preparing and using a variable epitope library may include preparing the variable epitope library (VEL), injecting the library into a subject and inducing an immune response in the subject against the VEL. In accordance with this embodiment, preparing a VEL may include preparing a VEL bearing epitopes of a pathogen-specific polypeptide. In another embodiment, the method may include preparing a VEL where the VEL bears epitopes of a disease-specific polypeptide. In one particular example, inducing an immune response in a subject may include inducing an immune response effective to protect a subject against infection with a pathogen. In another particular example, inducing the immune response may include inducing the immune response effective to treat a subject infected with a pathogen or to protect the subject against a disease such as cancer.

### DETAILED DESCRIPTION

In the following section, several methods are described to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description in order to prevent unnecessary masking of the various embodiments.

The present invention provides for VELs compositions and methods of use for treatment of disease. In one embodiment of the present invention, a composition may include a synthetic peptide. In accordance with this embodiment, the synthetic peptide may include at least one epitope of a pathogen- or disease-specific polypeptide, where at least one amino acid residue of the peptide is substituted with each of the other nineteen common amino acid residues.

### Variable Epitope Libraries (VELs)

The genetic variability of many pathogens and disease-related antigens results in the selection of mutated epitope variants able to escape control by immune responses. This is a major obstacle to vaccine development. The present invention relates to immunogens composed of epitope libraries derived from pathogens and disease-related antigens with genetic/antigenic variability.

The immunogen composed of epitope libraries is termed a variable epitope library (VEL). The VELs are composed of 8-50 amino acid (aa) length pathogen- or disease-related peptides P₁P₂P₃ . . . Pn. The numbers are positions (P) of wild type aa sequences, where “n” represents peptide length and the position of the last aa. In various embodiments of the invention, at least one aa and as many as 90% of wild type aa residues are randomly replaced by any aa of 20 possible aa residues. In alternative embodiments, the VELs may contain 30-120 aa recombinant peptides/polypeptides.

For example the composition of an exemplary VEL based on a hypothetical decapeptide P₁P₂P₃P₄P₅P₆P₇P₈P₉P₁₀ can be represented as P₁X₂P₃X₄P₅X₆P₇X₈P₉X₁₀ where X is any of 20 aa (amino acids) and P₁,P₃,P₅,P₇,P₉ are wild type aa sequences. Similarly, another version of VEL based on the same decapeptide may be constructed by replacing wild type aa residues by X residues at odd positions and leaving this time wild type residues at even positions. While in these two particular decapeptide-based VELs each individual library member has 50% of wild type and 50% of random aa residues, this proportion may be varied in such a manner that only one aa or up to 90% of wild type sequence will be replaced by random aa residues.

The complexities of VELs can be 20 epitope variants when only one aa is replaced in the epitope by random aa residues and up to about 10⁹ when several aa residues are simultaneously mutated. Since the appearance of any aa other than wild type aa within the epitopes derived from genetically variable pathogens or disease-related antigens including, for example, HIV, hepatitis A/B/C, rhinovirus, influenza virus, plasmodium falciparum, or some tumor antigens, is a frequent phenomenon, the VEL-based immunogen construction reflects antigenic diversity observed during the infection with the above mentioned pathogens and/or in diseases. Hence, use of VEL immunogens permits the generation of novel prophylactic and therapeutic vaccines capable of inducing a broad range of protective immune responses before the appearance of mutated epitopes (before infection) or when the amounts of mutated epitopes are low (early stages of infection and/or disease progression).

VELs may be generated based on defined pathogen or disease-related antigen-derived cytotoxic T lymphocyte (CTL), helper T lymphocyte (Th) or B lymphocyte epitopes and particularly, on epitopes derived from antigenically variable or relatively conserved regions of protein. Alternatively, the VELs may be built based on up to 50 aa long peptide regions of antigens containing clusters of epitopes. An individual VEL may contain: [1] variants of one CTL, Th or B cell epitope; [2] variants of several different CTL, Th or B cell epitopes; [3] any combination of these mutated CTL, Th and B cell epitopes expressed in a single up to 120 aa long artificial recombinant polypeptide; [4] up to 50 aa long mutated wild type-related peptide carrying several CTL, Th and/or B cell epitopes. Additionally, the VELs may be built based on 8-50 aa peptides selected from antigenically variable or relatively conserved regions of pathogen- or disease-related proteins without a prior knowledge of the existence of epitopes in these peptide regions. The candidate epitopes may be selected from scientific literature or from public databases. In preferred embodiments it may be particularly useful to include CTL epitopes in VELs, since the escape from protective CTL responses is an important mechanism for immune evasion by many pathogens, for example HIV and SIV.

VELs may take the form of DNA constructs, recombinant polypeptides or synthetic peptides and may be generated using standard molecular biology or peptide synthesis techniques, as discussed below. For example to generate a DNA fragment encoding particular epitope variants bearing peptides, a synthetic 40-70 nucleotide (nt) long oligonucleotide (oligo) carrying one or more random α-coding degenerate nucleotide triplet(s) may be designed and produced. The epitope-coding region of this oligo (oligol) may contain non-randomized 9-15 nt segments at 5′ and 3′ flanking regions that may or may not encode natural epitope-flanking 3-5 aa residues. Then, 2 oligos that overlap at 5′ and 3′ flanking regions of oligol and carry nt sequences recognized by hypothetical restriction enzymes A and B, respectively, may be synthesized and after annealing reaction with oligol used in a PCR. This PCR amplification will result in mutated epitope library-encoding DNA fragments that after digestion with A and B restriction enzymes may be combined in a ligation reaction with corresponding bacterial, viral or eukaryotic cloning/expression vector DNA digested with the same enzymes. The ligation mixtures may be used to transform bacterial cells to generate the VEL and then expressed as a plasmid DNA construct, in a mammalian virus or as a recombinant polypeptide. This DNA may also be cloned in bacteriophage, bacterial or yeast display vectors, allowing the generation of recombinant microorganisms.

In a similar manner, DNA fragments encoding VELs bearing 30-150 aa long peptides/polypeptides containing various combinations of 2-15 different mutated epitope variants may be generated using sets of 4-12 40-80 nt long overlapping oligos and a pair of oligos carrying restriction enzyme recognition sites and overlapping with adjacent epitope-coding oligos at 5′ and 3′ flanking regions. These oligos may be combined, annealed and used in a PCR assembly and amplification reactions. The resulting DNAs may be similarly cloned in the above mentioned vectors.

In another embodiment, DNAs coding for mutated epitope clusters may also be obtained using pairs of wild type sequence-specific oligos carrying DNA restriction sites and pathogen- or antigen-derived genomic or cDNA as template in a PCR with an error-prone DNA polymerase. These DNAs also may be cloned in corresponding vectors. The VELs may be expressed in mammalian virus vectors, such as modified Vaccinia ankara, an adenoviral, a canary pox vectors, produced as recombinant polypeptides or as recombinant microorganisms and used individually as immunogens or may be combined and used as a mixture of VELs.

In one example, synthetic peptide libraries representing VELs and varying in length from 7 to 50 aa residues may be generated by solid phase Fmoc peptide synthesis technique where in a coupling step equimolar mixtures of all proteogeneic aa residues may be used to obtain randomized aa positions. This technique permits the introduction of one or more randomized sequence positions in selected epitope sequences and the generation of VELs with complexities of up to 10⁹.

In one embodiment, vaccine compositions containing one or more VELs may be formulated with a pharmaceutically acceptable carrier or adjuvant, and administered to an animal or to a patient. Other approaches for the construction of VELs, expression and/or display vectors, optimum vaccine composition, routes for vaccine delivery and dosing regimes capable of inducing prophylactic and therapeutic benefits may be determined by one skilled in the art. The immunogens based on VEL(s) are useful for inducing protective immune responses against pathogens and tumors with antigenic variability, as well as may be effective in modulating allergy, inflammatory and autoimmune diseases.

The skilled artisan will realize that in alternative embodiments, less than the 20 naturally occurring amino acids may be used in a randomization process. For example, certain residues that are known to be disruptive to protein or peptide secondary structure, such as proline residues, may be less preferred for the randomization process. VELs may be generated with the 20 normal aa residues or with some subset of the 20 normal aa residues.

In various embodiments, in addition to or in place of the 20 naturally occurring aa residues, the VELs may contain at least one modified or unusual amino acid, including but not limited to those shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

VELs may be made by any technique known to those of skill in the art, including the expression of polypeptides or peptides through standard molecular biological techniques or the chemical synthesis of peptides. The nucleotide and polypeptide and peptide sequences corresponding to various pathogen- or disease-related antigens are known in the art and may be found at computerized databases known to those of ordinary skill in the art. One such database is the National Center for Biotechnology Information's Genbank and GenPept databases. Any such known antigenic sequence may be used in the practice of the claimed methods and compositions.

### Combinatorial Libraries

Combinatorial libraries of such compounds or of such targets can be categorized into three main categories. The first category relates to the matrix or platform on which the library is displayed and/or constructed. For example, combinatorial libraries can be provided (i) on a surface of a chemical solid support, such as microparticles, beads or a flat platform; (ii) displayed by a biological source (e.g., bacteria or phage); and (iii) contained within a solution. In addition, three dimensional structures of various computer generated combinatorial molecules can be screened via computational methods.

Combinatorial libraries can be further categorized according to the type of molecules represented in the library, which can include, (i) small chemical molecules; (ii) nucleic acids (DNA, RNA, etc.); (iii) peptides or proteins; and (iv) carbohydrates.

The third category of combinatorial libraries relates to the method by which the compounds or targets are synthesized, such synthesis is typically effected by: (i) in situ chemical synthesis; (ii) in vivo synthesis via molecular cloning; (iii) in vitro biosynthesis by purified enzymes or extracts from microorganisms; and (iv) in silico by dedicated computer algorithms.

Combinatorial libraries indicated by any of the above synthesis methods can be further characterized by: (i) split or parallel modes of synthesis; (ii) molecules size and complexity; (iii) technology of screening; and (iv) rank of automation in preparation/screening.

The complexity of molecules in a combinatorial library depends upon the diversity of the primary building blocks and possible combinations thereof. Furthermore, several additional parameters can also determine the complexity of a combinatorial library. These parameters include (i) the molecular size of the final synthesis product (e.g., oligomer or small chemical molecule); (ii) the number of bonds that are created in each synthesis step (e.g., one bond vs. several specific bonds at a time); (iii) the number of distinct synthesis steps employed; and (iv) the structural complexity of the final product (e.g., linear vs. branched molecules).

Combinatorial libraries can be synthesized of several types of primary molecules, including, but not limited to, nucleic and amino acids and carbohydrates. Due to their inherent single bond type complexity, synthesizing nucleic and amino acid combinatorial libraries typically necessitates only one type of synthesis reaction. On the other hand, due to their inherent bond type complexity, synthesizing complex carbohydrate combinatorial libraries necessitates a plurality of distinct synthesis reactions.

### Synthetic Peptides

The VELs of the invention may be synthesized, in whole or in part, in solution or on a solid support in accordance with conventional techniques. Various automatic synthesizers are commercially available and can be used in accordance with known protocols. See, for example, Stewart and Young, (Solid Phase Peptide Synthesis, 2d. ed., Pierce Chemical Co., 1984); Tam et al., (J. Am. Chem. Soc., 105:6442, 1983); Merrifield, (Science, 232: 341-347, 1986); and Barany and Merrifield (The Peptides, Gross and Meienhofer, eds., Academic Press, New York, pp. 1-284, 1979) each incorporated herein by reference. Short peptide sequences, usually from about 6 up to about 35 to 50 amino acids, can be readily synthesized by such methods. A common method of peptide synthesis involves phosphoramidite based chemistry using commercial peptide synthesizers, such as available from Applied Biosystems (Foster City, Calif.). Typically, a cartridge based system includes a separate cartridge for each amino acid to be sequentially incorporated into the peptide. For incorporation of the substituted amino acid residues of the VELs, a cartridge containing a mixture of all 20 amino acids may be utilized. Such synthetic peptides may also be purchased from known commercial sources (e.g., Midland Certified Reagents, Midland, Tex.). Alternatively, recombinant DNA technology may be employed wherein a nucleotide sequence which encodes a peptide of the invention is inserted into an expression vector, transformed or transfected into an appropriate host cell, and cultivated under conditions suitable for expression as discussed below.

### Expression of Proteins or Peptides

In certain embodiments, it may be preferred to make and use an expression vector that encodes and expresses a particular VEL. Gene sequences encoding various polypeptides or peptides may be obtained from GenBank and other standard sources, as disclosed above. Expression vectors containing genes encoding a variety of known proteins may be obtained from standard sources, such as the American Type Culture Collection (Manassas, Va.). For relatively short VELs, it is within the skill in the art to design synthetic DNA sequences encoding a specified amino acid sequence, using a standard codon table, as discussed above. Genes may be optimized for expression in a particular species of host cell by utilizing well-known codon frequency tables for the desired species. Genes may represent genomic DNA sequences, containing both introns and exons, or more preferably comprise cDNA sequences, without introns.

Regardless of the source, a coding DNA sequence of interest can be inserted into an appropriate expression system. The DNA can be expressed in any number of different recombinant DNA expression systems to generate large amounts of the polypeptide product, which can then be purified and used in various embodiments of the present invention.

Examples of expression systems known to the skilled practitioner in the art include bacteria such as E. Coli, yeast such as Pichia pastoris, baculovirus, and mammalian expression systems such as in Cos or CHO cells. Expression is not limited to single cells, but may also include protein production in genetically engineered transgenic animals, such as rats, cows or goats. A complete gene can be expressed or, alternatively, fragments of the gene encoding portions of polypeptide can be produced.

In certain broad applications of the invention, the sequence encoding the polypeptide may be analyzed to detect putative transmembrane sequences. Such sequences are typically very hydrophobic and are readily detected by the use of standard sequence analysis software, such as MacVector (IBI, New Haven, Conn.). The presence of transmembrane sequences may be deleterious when a recombinant protein is synthesized in many expression systems, especially E. coli, as it leads to the production of insoluble aggregates which are difficult to renature into the native conformation of the protein. Deletion of transmembrane sequences typically does not significantly alter the conformation of the remaining protein structure. Deletion of transmembrane-encoding sequences from the genes used for expression can be achieved by standard techniques. For example, fortuitously-placed restriction enzyme sites can be used to excise the desired gene fragment, or PCR-type amplification can be used to amplify only the desired part of the gene.

The gene or gene fragment encoding a polypeptide may be inserted into an expression vector by standard subcloning techniques. An E. coli expression vector may be used which produces the recombinant polypeptide as a fusion protein, allowing rapid affinity purification of the protein. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these systems produce recombinant polypeptides bearing only a small number of additional amino acids, which are unlikely to affect the activity or binding properties of the recombinant polypeptide. For example, both the FLAG system and the 6xHis system add only short sequences, both of which have no adverse affect on folding of the polypeptide to its native conformation. Other fusion systems are designed to produce fusions wherein the fusion partner is easily excised from the desired polypeptide. In one embodiment, the fusion partner is linked to the recombinant polypeptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

The expression system used may also be one driven by the baculovirus polyhedron promoter. The gene encoding the polypeptide may be manipulated by standard techniques in order to facilitate cloning into the baculovirus vector. One baculovirus vector is the pBlueBac vector (Invitrogen, Sorrento, Calif.). The vector carrying the gene for the polypeptide is transfected into Spodoptera frugiperda (Sf9) cells by standard protocols, and the cells are cultured and processed to produce the recombinant protein. See Summers et al., A Manual of Methods for Baculovirus Vectors and Insect Cell Culture Procedures, Texas Agricultural Experimental Station; U.S. Pat. No. 4,215,051.

To express a recombinant encoded protein or peptide, whether mutant or wild-type, one would prepare an expression vector that comprises one of the isolated nucleic acids under the control of, or operatively linked to, one or more promoters. To bring a coding sequence “under the control of” a promoter, one positions the 5′ end of the transcription initiation site of the transcriptional reading frame generally between about 1 and about 50 nucleotides “downstream” (i.e., 3′) of the chosen promoter. The “upstream” promoter stimulates transcription of the DNA and promotes expression of the encoded recombinant protein.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which may be used by the microbial organism for expression of its own proteins.

In addition, phage vectors containing replicon and control sequences that are compatible with the host microorganism may be used as transforming vectors in connection with these hosts. For example, the phage lambda GEM™-1 may be utilized in making a recombinant phage vector which may be used to transform host cells, such as E. coli LE392.

Further useful vectors include pIN vectors and pGEX vectors, for use in generating glutathione S-transferase (GST) soluble fusion proteins for later purification and separation or cleavage. Other suitable fusion proteins are those with β-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid already contains the trp1 gene which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autographa californica nuclear polyhidrosis virus (AcNPV) is used as a vector to express foreign genes. The virus grows in Spodoptera frugiperda cells. The isolated nucleic acid coding sequences are cloned into non-essential regions (for example the polyhedrin gene) of the virus and placed under control of an AcNPV promoter (for example the polyhedrin promoter). Successful insertion of the coding sequences results in the inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, 3T3, RIN and MDCK cell lines. In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cells lines or host systems may be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter) as known in the art.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the Hind III site toward the Bgl I site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals known in the art may also be required for efficient translation of the claimed isolated nucleic acid coding sequences. One of ordinary skill in the art would readily be capable of determining this and providing the necessary signals

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins by stable expression known in the art may be required.

A number of selection systems may be used, including but not limited to, the herpes simplex virus thymidine kinase, hypoxanthine-guanine phosphoribosyltransferase and adenine phosphoribosyltransferase genes, in tk-, hgprt- or aprt-cells, respectively. Also, antimetabolite resistance may be used as the basis of selection for dhfr, that confers resistance to methotrexate; gpt, that confers resistance to mycophenolic acid; neo, that confers resistance to the aminoglycoside G-418; and hygro, that confers resistance to hygromycin. These and other selection genes may be obtained in vectors from, for example, ATCC or may be purchased from a number of commercial sources known in the art (e.g., Stratagene, La Jolla, Calif.; Promega, Madison, Wis.).

Where substitutions into naturally occurring pathogen- or disease-related polypeptide sequences are desired, the nucleic acid sequences encoding the native polypeptide sequence may be manipulated by well-known techniques, such as site-directed mutagenesis or by chemical synthesis of short oligonucleotides followed by restriction endonuclease digestion and insertion into a vector, by PCR based incorporation methods, or any similar method known in the art.

### Protein Purification

In certain embodiments a polypeptide or peptide may be isolated or purified. Protein purification techniques are well known to those of skill in the art. These techniques involve, at one level, the homogenization and crude fractionation of the cells, tissue or organ to polypeptide and non-polypeptide fractions. The peptide or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of protein purification by affinity chromatography is disclosed in U.S. Pat. No. 5,206,347. A particularly efficient method of purifying peptides is fast performance liquid chromatography (FPLC) or even HPLC.

A purified polypeptide or peptide is intended to refer to a composition, isolatable from other components, wherein the polypeptide or peptide is purified to any degree relative to its naturally-obtainable state. An isolated or purified polypeptide or peptide, therefore, also refers to a polypeptide or peptide free from the environment in which it may naturally occur. Generally, “purified” will refer to a polypeptide or peptide composition that has been subjected to fractionation to remove various other components. Where the term “substantially purified” is used, this designation will refer to a composition in which the polypeptide or peptide forms the major component of the composition, such as constituting about 50%, about 60%, about 70%, about 80%, about 90%, about 95%, or more of the polypeptides in the composition. Various methods for quantifying the degree of purification of the polypeptide or peptide are known to those of skill in the art in light of the present disclosure. These include, for example, assessing the amount of polypeptides within a fraction by SDS/PAGE analysis.

Various techniques suitable for use in protein purification are contemplated herein and are well known. There is no general requirement that the polypeptide or peptide always be provided in their most purified state. Indeed, it is contemplated that less substantially purified products will have utility in certain embodiments

In another embodiment, affinity chromatography may be required and any means known in the art is contemplated herein.

### Formulations and Routes for Administration to Patients

Where clinical applications are contemplated, it will be necessary to prepare pharmaceutical compositions—i.e. VEL compositions—in a form appropriate for the intended application. Generally, this will entail preparing compositions that are essentially free of impurities that could be harmful to humans or animals.

One generally will desire to employ appropriate salts and buffers to render polypeptides stable and allow for uptake by target cells. Aqueous compositions may comprise an effective amount of polypeptide dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the polypeptides of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal, intraarterial or intravenous injection. Such compositions normally would be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Procedures that are constructively reduced to practice (or prophetic examples) are described in the present tense, and procedures that have been carried out in the laboratory are set forth in the past tense.

### Example 1

### VELs Against Human Immunodeficiency Virus Coat Protein

In an exemplary embodiment, VELs capable of inducing an immune response against the Human Immunodeficiency Virus (HIV) gp120 coat protein are prepared. Different epitopic domains of gp120 and/or the gp160 precursor protein have been reported in the literature (e.g., Thali et al., 1991, J. Virol. 65:6188-93) and are known in the art and any such known epitope may be used. For example, an epitope comprising Thr297, Phe383, Tyr384, Arg419, Ile240, Leu240, Thr415, Leu416, Pro417, Lys421 and Trp112 has been reported. A polypeptide comprising gp120 residues 383-421 is prepared by chemical synthesis, with amino acid substitutions. In one embodiment, residues Phe383, Tyr384, Thr415, Leu416, Pro417, Arg419 and Lys421 are maintained invariant and the other residues 385-414, 418 and 420 are varied, with all 20 amino acids substituted into those positions. In another embodiment, all even numbered residues are maintained invariant and all odd numbered residues are substituted with each of the 20 aa residues. In yet another embodiment, all odd numbered residues are maintained invariant and all even numbered residues are varied.

Another reported gp120 epitope is comprised of residues 429-443. A VEL is prepared against this sequence by chemical synthesis of a synthetic peptide. In one embodiment, every odd numbered residue is held invariant and the even numbered residues are substituted with each of the 20 amino acids. In another embodiment, residues 430-443 are held invariant and residue 429 is substituted. In yet another embodiment, residues 429-434 are held invariant. In the remaining residues 435-443, even numbered residues are substituted and odd numbered residues are held invariant.

Another reported gp120 epitope is comprised of residues 470-484. In one embodiment, a synthetic peptide is constructed with all even numbered residues of 470-484 held invariant and all odd numbered residues substituted.

In yet another exemplary embodiment, a VEL comprising a mixture of synthetic peptides to residues 383-421, 429-443 and 470-484, substituted as described above, is prepared.

The VELs are injected into a subject, such as a mouse, rabbit, cat, chimpanzee, rhesus monkey, or human. The toxicity, distribution, localization and elimination of the VELs is determined. Injection of VEL, tailored against the coat protein of SIV, is demonstrated to provide efficacy against SIV infection in chimpanzees. Injection of VELs prepared against the HIV gp120 coat protein epitopes is demonstrated to provide efficacy against HIV infection.

### Example 2

In one exemplary study, immunogens are generated based on VEL vaccine concept and will be tested for induction of broad T cell immune responses in mice. Here, VEL-based vaccine concept will be tested for immunogens bearing single HIV-1 CTL epitope libraries in conventional mice and later in HLA transgenic mice. The immunogens carrying CTL epitopes will be generated as synthetic peptides, DNA vaccine constructs and recombinant M13 phages in different molecular contexts. Then multiepitope DNA, eukaryotic viral vector, recombinant protein and recombinant M13 vaccines will be generated by combining 10-12 CTL, Th and/or B cell epitopes and their variants bearing libraries in a single polypeptide to test efficacy in monkeys (including SIV-derived epitopes in VEL-based vaccines). Finally, these tests will be performed in humans.

Using these techniques, vaccines may be made by combining several such multiepitope polypeptides containing in sum many epitope variant libraries (30-60 VEL-based epitope libraries) for one or more vaccine preparations or for a single vaccine preparation.

In another example, similar to outlined above, immunogens may be generated by introducing random amino acid sequences at 1, 2, or 3 positions within pathogen- or disease-derived epitopes and used alone or in combination with several other VEL-based immunogens as vaccine components.

### Methods

### Design and Construction of VEL-Based Immunogens

### Synthetic Peptides

In one exemplary method, synthetic peptides corresponding to HIV-1 optimal CTL epitopes were prepared (e.g. Invitrogen (Table 2)). For example, gp120 V3-derived peptide L (aa 311-320; RGPGRAFVTI: SEQ ID NO:1) and Gag-derived peptide GP (aa 65-73; AMQMLKETI SEQ ID NO:2) restricted by BALB/c H2-D^{d} and H2-K^{d} (respectively), have been derived. In one example, the corresponding synthetic peptide libraries of VELs based on these epitope sequences, may be SLVEL1 SEQ ID NO:3, SLVEL2 SEQ ID NO:4, SGPVEL1 SEQ ID NO:5 and SGPVEL2 SEQ ID NO:6. These libraries were synthesized at GenScript Corp. as combinatorial peptide libraries. In one example, libraries with 5 randomized amino acid positions containing around 3.2×10⁶ individual peptides (SLVEL1 SEQ ID NO:3 and SLVEL2 SEQ ID NO:4) and libraries with 4 randomized amino acid positions containing 1.6×10⁵ peptides (SGPVEL1 SEQ ID NO:5 and SGPVEL2 SEQ ID NO:6), respectively (Table 2.) were generated. The amino acid positions of epitopes within epitope libraries marked as X are positions where any natural amino acid out of the 20 common amino acids may appear randomly.

### DNA Constructs and Recombinant M13 Phages

In general, molecular biology procedures may be carried out using standard protocols known in the art or as recommended by manufacturers. Restriction enzymes, DNA isolation/purification kits, T4 DNA ligase, calf intestine alkaline phosphatase (CIAP) and M13KO7 helper phage can be obtained for example from Invitrogen (Carlsbad, Calif., USA), Qiagen (Valencia, Calif., USA) or GibcoBRL (Rockville, Md., USA).

In one exemplary method, DNA constructs expressing HIV-1-derived CTL epitopes may be generated by inserting the epitopes into human immunoglobulin (Ig) heavy-chain variable (V_{H}) domain by replacing complementarity-determining-region 3 (HCDR3) of V_{H} by CTL epitopes/peptides (Manoutcharian K., et al. Phage-displayed T-cell epitope grafted into immunoglobulin heavy-chain complementarity-determining regions: an effective vaccine design tested in murine cysticercosis. Infect. and Immunity. 1999; 67(9):4764-4770, incorporated herein by reference in its entirety). In one example, to generate a wild-type (WT) Ig V_{H} domain, a set of partially overlapping oligonucleotides collectively coding for the framework (FR) and CDR regions of the human Ig V_{H} domain DP47 (Oligos B1-B8, Table 2) was synthesized (for example by Operon Technologies, Inc., Alameda, Calif.). Oligonucleotides B1 to B8 (for example: 4 pmol each; the overlaps between the complementary oligonucleotides are 12 to 20 nucleotides) were combined and assembled in PCR with Pfu DNA polymerase (Stratagene, La Jolla, Calif.) by cycling the reaction mixture (around 50 μl) 30 times (95° C. for 2 min; 56° C. for 2 min; 72° C. for 1 min). An aliquot from this reaction (approximately 5 μl), containing a 350-bp DNA fragment coding for the WT Ig V_{H} domain was amplified by polymerase chain reaction (PCR) (for example, 50 μl) by cycling 30 times (94° C. for 1 min; 65° C. for 1 min; 72° C. for 1 min) with the 5 NAmp and 3 NAmp primers (30 pmol each), which introduce PstI and Bst EII restriction sites at the 5′ and 3′ ends of the synthesized Ig V_{H} domain, respectively (the restriction sites are underlined in the oligos, Table 2). The assembly and amplification of PCR products were checked by agarose gel electrophoresis, and the DNA of the engineered V_{H} domains, after purification from the gel with a for example by a Master Kit (Bio-Rad Laboratories, Hercules, Calif.), cut with PstI and BstEII (Stratagene) and purified again. Then, 1 μg of this DNA was ligated with 10 U of T4 DNA ligase (Amersham-Life Science, Cleveland, Ohio) to approximately 1 μg of PstI- and BstEII-digested DNA of the VHExpress eukaryotic expression vector (Persic L., et al. An integrated vector system for the eukaryotic expression of antibodies or their fragments after selection from phage display libraries. Gene. 1997; 10; 187(1):9-18 incorporated herein by reference). The ligated DNA was column purified and used to transform Escerichia coli TG1 cells by electroporation using Gene Pulser II System (Bio-Rad Laboratories, Inc., Hercules, Calif., USA). PCR assembly and cloning were verified by dideoxy sequencing with [α-³⁵S]dATP (Amersham) and the T7 Sequenase Quick-Denature plasmid sequencing kit (Amersham).

In another example, to generate modified V_{H} domains expressing CTL epitopes and epitope libraries, the same mixture of oligos B1-B8 were used in PCRs by replacing B7 oligo coding for CDR3 region with oligos LN, L1 or L2 coding for WT L epitope, LVEL1 or LVEL2, respectively, and using the same 5 NAmp and 3 NAmp primers as described previously. To generate epitope variant libraries, degenerate oligos L1, L2, GP1 and GP2 (where K in NNK triplets are T or C nucleotide) were used. To construct VEL-expressing DNA vectors, ten electroporations were performed using the ligation mixtures, and the transformed TG1 cells were plated on LB-Amp plates to determine the diversity of the libraries. In another similar example, modified V_{H} domains carrying Gag-derived GP CTL epitopes were generated and cloned in VHExpress vector. Libraries with complexities of about 1-3×10⁶ members for L epitope and libraries of 1-2×10⁵ complexities for GP epitope are expected using these procedures. The plasmid DNA was produced by growth in Escherichia coli (strain TG1) in Terrific Broth with Ampicillin (50 μg/ml) and purified for example using Qiagen MegaPrep columns, according to the manufacturer's directions (Qiagen, Valencia, Calif.).

In one exemplary method, to express the L and GP CTL epitopes and epitope variant libraries on M13 phage surface as fusions with major phage coat protein (cpVIII) at high copies, the corresponding DNA fragments have been cloned in pG8SAET phagemid vector (K. Jacobsson and L. Frykberg 2001. Shotgun phage display cloning. Comb. Chem. High. Throughput Screen. 4:135-143, incorporated herein by reference in its entirety). This time the epitopes are not in the context of V_{H} domain, the epitopes are flanked by 5 amino acids from FR3 and FR4 and, in the case of GP epitope there are also 2 flanking amino acids derived from natural HIV-1 epitope flanking regions. First, DNA fragments can be generated by PCR using oligos LN, L1 or L2 coding for WT L epitope, LVEL1 SEQ ID NO:7 or LVEL2 SEQ ID NO:8, respectively, and the primers 5 DAmp/3 DAmp carrying NcoI and Bam HI restriction sites (underlined in oligos, Table 2). Then, these DNAs were purified and used in separate ligation reactions with the DNA of similarly digested phagemid vector DNA as described above. After electroporation, the transformed TG-1 cells were plated on LB-Amp plated to determine the diversities of the libraries. L and GP epitope-based phage-displayed libraries were generated of about 1-3×10⁶ and 1-2×10⁵ members, respectively. The resultant phagemid libraries were rescued and amplified using M13KO7 helper phage Then purified by double PEG/NaCl (20% w/v polyethylene glycol 1-8000; 2.5 M NaCl) precipitation and resuspended in Tris-buffered saline (TBS). The typical phage yields were 10¹⁰-10¹¹ colony-forming units (cfu) per milliliter of culture medium. The generated recombinant phage particles have been be used as immunogens/antigens in immunization and lymphoproliferation assays. Twenty phage displayed epitope variants were randomly selected from each epitope library (LVEL1 SEQ ID NO:7, LVEL2 SEQ ID NO:8, GPVEL1 SEQ ID NO:9 and GPVEL2 SEQ ID NO:10) and used as antigens in T cell activation assays. In addition, the DNA from these phage clones were sequenced and corresponding peptide inserts were prepared as synthetic peptides (20 peptides for each epitope library) and similarly used as antigens in T cell assays.

### VEL-Based Vaccine Immunogenicity Testing in Mice.

### Mice and Immunizations

In one exemplary method, the immune responses induced by different immunogens carrying VEL antigens were evaluated in groups of 8-10 female BALB/c and C57BL/6 mice, 6 to 8 weeks old, were used. Direct assessment of epitope immunogenicity was completed using synthetic peptides, 50 μg/dose emulsified in IFA which were administered s.c. to mice. When the DNA vaccine was used, groups of mice were immunized bilaterally with 100 μg of DNA into tibialis anterior muscle, which was pretreated by cardiotoxin injection. 2×10¹⁰ recombinant M13 phage particles were used to immunize mice by subcutaneous injection. In addition, the groups of mice were immunized with DNA expressing wild-type Ig V_{H} domain, non-related phage and synthetic peptides for controls. All mice were immunized by single injection or primed by DNA and boosted with synthetic peptides or M13 phages 14 days after the priming. Separately, the mice were immunized with plasmid DNA constructs and recombinant phages carrying sublibraries of VELs with different levels of complexities (1×10³, 5×10³, 2×10⁴, or 1×10⁵ individual members). These sublibraries were obtained by plating the dilutions of DNA constructs-harboring bacterial stocks as colonies or phage particles on LB-Amp plates and isolating plasmid DNA or phage sublibraries, respectively, as described above. In one example, two related assays were used to measure CTL activity induced by immunization in mice, an ELISPOT and a chromium release assay.

### IFN-γ ELISPOT Assay

In one example, an enzyme-linked immunospot (ELISPOT) assay was performed to measure gamma interferon (IFN-γ) production. Briefly, 96-well multiscreen HA plates (Millipore) were coated by overnight incubation (100 μl/well) at 4° C. with rat anti-mouse IFN-γ MAb (clone R4-6A2; BD Pharmingen) at 10 μg/ml in PBS. Splenocytes were harvested from individual mice 1 week after immunization. Effector cells were plated in triplicate at 2×10⁵/well in a 100-μl final volume with medium alone, 4 μg of epitope peptide or 5×10¹⁰ phage particles per ml. As negative controls, L-derived phage-displayed variant epitopes and corresponding synthetic peptides were used to analyze the spleen cells from mice immunized with GP epitopes and variant epitope libraries and vice versa. After a 24-h incubation at 37° C., the plates were washed free of cells with PBS-0.05% Tween 20 and incubated overnight at 4° C. with 100 μl of biotinylated rat anti-mouse IFN-γ MAb (clone XMG1.2; BD Pharmingen) per well at 5 μg/ml. Plates were washed four times, and 75 μl of streptavidin-alkaline phosphatase (Southern Biotechnology Associates) was added at a 1/500 dilution. After a 2-h incubation, plates were washed four times and developed with Nitro Blue Tetrazolium-5-bromo-4-chloro-3-indolylphosphate chromogen (Pierce). Plates were analyzed with an ELISPOT reader (Hitech Instruments).

### ⁵¹Chromium Release Assay

in a 24-well plate (8×10⁶/well) with 10 ng of epitope peptide or 10¹⁰ phage particles per ml previously selected in ELISPOT assay as antigens capable of stimulating T cells . Interleukin-2 (IL-2) (Sigma) was added to cultures on day 2 to a final concentration of 10 U/ml. On day 7, cells were harvested, washed once, and used as effectors in a ⁵¹Cr release assay with P815 target cells (American Type Culture Collection). P815 cells were cultured overnight in the presence of medium alone, with 100 ng of synthetic peptide or 10¹⁰ phage particles per ml. Cells (2×10⁶) were labeled with 150 μCi of ⁵¹Cr for 1 h at 37° C., washed twice, and added to a 96-well round-bottom plate at 10⁴/well in 100 μl of 10% RPMI medium. Titrations of effector cells were added to triplicate wells in 100 μl of medium. Lytic activity was assessed in a standard 4-h ⁵¹Cr release assay. Percent specific lysis was calculated as follows: 100×(experimental−spontaneous release)/(maximum−spontaneous release).

### Flow Cytometric Analysis

In another exemplary method for phenotyping the CTL epitope-specific CD8⁺ T cells, splenocytes were sampled 1 week after immunization of mice and stained with anti-CD8α MAb (53-6.7; BD Pharmingen) conjugated with peridinin chlorophyll protein-Cy5.5, anti-CD62L MAb (MEL-14; BD Pharmingen) conjugated with APC, anti-CD44 MAb (IM-7; eBiosciences) conjugated with APC-Cy7, anti-CD127 MAb (A7R34; eBiosciences) conjugated with PE-Cy7. Multicolor flow analysis was performed using the BD LSRII Cytometer (BD Biosciences) and the FlowJo software (Tree Star).

### Statistical Analysis

Data were expressed as means±standard errors of the means (SEM). Statistical tests were performed using Student's t test. A P value of less than 0.05 was considered significant.

### Analysis

The simultaneous presentation of thousands of epitope variants to immune system after vaccination with VEL-based immunogens induce the activation of broad range of T cells (both CTL and Th). These T cells are capable of recognizing both the pathogen's epitopes present at the time of experimental or natural pathogen challenge and the variants of these epitopes that appear rapidly upon infection. In a naïve host, this induces a large pool of effector and memory T cells capable of containing or clearing the infecting pathogen (prophylactic vaccine). This vaccine is able to reactivate memory T cells and/or induce de novo responses against existing or newly evolving variant epitopes, respectively, in infected individuals (therapeutic vaccine).

VELs were generated based on two HIV-1 Env- and Gag-derived CTL epitopes. The immunogens consist of optimal/minimal CTL epitopes as well as the libraries of their variants (VELs) designed and generated as synthetic peptides, DNA constructs or recombinant M13 bacteriophages in various molecular contexts (see Table 2. and Protocols), (HIV-1 CTL minimal epitope and corresponding VELs have been generated as synthetic peptides, DNA constructs and M13 phages. Also, DNA constructs and recombinant M13 phages expressing the CTL epitopes and VELs in the context of Ig V_{H} were generated). For lymphoproliferation assays 20 antigens representing variant epitopes in the form of synthetic peptides and recombinant phages were prepared by randomly selecting 20 individual phage clones each expressing defined epitope variant from phage-displayed epitope libraries.

### Data in Mice

In one example, to obtain experimental data supporting these disclosed vaccine concepts, mice are immunized with various vaccine compositions carrying VELs using various immunization schemes. The induced T cell responses in mice are measured.

The activated spleen cells and CD8+ T cells from BALB/c mice immunized with immunogens carrying wild type CTL epitope recognize a few if any epitope variant(s) of the corresponding epitope in lymphoproliferation assays. The splenocytes from mice immunized with control non-related VEL or CTL epitope (Env-derived epitope and a set of variant epitopes serve as negative control antigens in T cell assays using spleen cells from mice immunized with Gag-derived epitope and epitope libraries and vice versa) in different forms and molecular contexts (synthetic peptide(s), DNA construct or recombinant phage) will not recognize corresponding epitope(s). Since both CTL epitopes included in immunogens have H-2^{d} restriction, T cell activation induced in BALB/c but not in C57BL/6 mice carrying H-2^{b} background.

By contrast, the splenocytes and the purified CD8+ T cells from BALB/c mice immunized with immunogens carrying VELs recognize more than 30% and up to 90% of corresponding variant epitopes along with the respective wild type epitope in lymphoproliferation assays. The spleen cells from similarly immunized C57BL/6 mice recognize the wild type and several variant epitopes (approximately 20%) due to the activation of a broad subset of T cells recognizing closely related epitopes as the result of multiple conformational changes (including MHC-anchor and TCR contact positions) within the epitope used for immunization.

In another example, epitope-specific CD8+ T cells are characterized by evaluating their state of maturation and functional commitment by measuring their expression of CD62L, CD127 and CD44. The majority of the cells are effector cells (CD44^{hi}, CD127⁻, and CD62^{lo}) (2-3 weeks post immunization) effector memory is induced (CD44^{hi}, CD127⁺, and CD62^{lo}) or central memory cells are induced (CD44^{hi}, CD127⁺, and CD62^{lo}). Various immunogens and immunization schedules during the period of up to one year after immunization are tested.

Alternatively, exemplary methods for determining minimally required complexities of VEL-containing immunogens capable of inducing the activation of a broadest range of T cells recognizing large number of CTL epitope variants are tested. T-cell responses in mice immunized with DNA and recombinant phage carrying VELs with different levels of complexities (1×10³, 5×10³, 2×10⁴, or 1×10⁵ individual members) are analyzed. The immunization of mice with VELs containing 5×10³ or 2×10⁴ epitope variants is sufficient to induce T cells specifically recognizing 30-90% of tested epitope variants.

TABLE 2
CONSTRUCTION OF IMMUNOGENS

| PEPTIDES/ | | |
|---|---|---|
| IMMUNOGENS | OLIGOS | CLONING VECTORS |
| FRAMEWORK 1 | B1 | EUKARYOTIC EXPRESSION |
| | 5′GAGGTGCAGCTGTTGGAGTCT | VECTOR |
| | GGGGGAGGCTTGGTACAGCCT | VHEXPRESS |
| | GGGGGGTCCCTGAGACTCTCCT | WILD-TYPE V_{H} EXPRESSED IN THE |
| | GTGCA3′ | CONTEXT OF IG HEAVY CHAIN |
| | SEQ ID NO: 11 | DNA CONSTRUCT |
| CDR1 | B2 | |
| | 5′CCCTGGAGCCTGGCGGACCC | |
| | AGCTCATGGCATAGCTGCTAAA | |
| | GGTGAATCCAGAGGCTGCACA | |
| | GGAGAGTCTCAGGGA3′ | |
| | SEQ ID NO: 12 | |
| FRAMEWORK 2 | B3 | |
| | 5′TGGGTCCGCCAGGCTCCAGG | |
| | GAAGGGGCTGGAGTGGGTCTC | |
| | A3′ | |
| | SEQ ID NO: 13 | |
| CDR2 | B4 | |
| | 5′GAACCGGCCCTTCACGGAGT | |
| | CTGCGTAGTATGTGCTACCACC | |
| | ACTACCACTAATAGCTGAGACC | |
| | CACTCCAGCCCCTT3′ | |
| | SEQ ID NO: 14 | |
| FRAMEWORK 3 | B5 | |
| | 5′GACTCCGTGAAGGGCCGGTT | |
| | CACCATCTCCAGAGACAATTCC | |
| | AAGAACACGCTGTATCTGCAAA | |
| | TGAAC3′ | |
| | SEQ ID NO: 15 | |
| FRAMEWORK 3/CDR3 | B6 | |
| | 5′CGCACAGTAATATACGGCCG | |
| | TGTCCTCGGCTCTCAGGCTGTT | |
| | CATTTGCAGATACAGCGT3′ | |
| | SEQ ID NO: 16 | |
| FRAMEWORK 3/CDR3 | B6 | |
| | 5′CGCACAGTAATATACGGCCG | |
| | TGTCCTCGGCTCTCAGGCTGTT | |
| | CATTTGCAGATACAGCGT3′ | |
| | SEQ ID NO: 16 | |
| CDR3 | B7 | |
| | 5′GCCGTATATTACTGTGCGAAA | |
| | GGTAGTTACTTTGACTACTGGG | |
| | GCCAGGGAACCCTGGTC3′ | |
| | SEQ ID NO: 17 | |
| FRAMEWORK 4 | B8 | |
| | 5′TGAGGAGACGGTGACCAGGG | |
| | TTCCCTGGCCCCA3′ | |
| | SEQ ID NO: 18 | |
| PRIMERS FOR PCR | 5NAMP | |
| AMPLIFICATION | 5′ATTCTAGCCATGGTGAATTC | |
| | CTGCAGGAGGTGCAGCTGTTGGA | |
| | GTGT3′ | |
| | SEQ ID NO: 19 | |
| PRIMERS FOR PCR | 3NAMP | |
| AMPLIFICATION | 5′CATGTACGTATGGATCCATTG | |
| | AGGAGACGGTGACCAGGGT 3′ | |
| | SEQ ID NO: 20 | |
| WILD-TYPE ENV EPITOPE | LN | VHEXPRESS |
| LWT | 5′GCC GTA TAT TAC TGT GCG | LWT EXPRESSED IN THE CONTEXT |
| GVYYGA RGPGRAFVTI | CGT GGT CCT GGT CGT GCT TTT | OF CDR3 OF V_{H} |
| WGQGT | GTT ACT ATT TGG GGC CAG | PHAGE DISPLAY VECTOR |
| | GGA ACC CTG 3′ | PG8SAET |
| | SEQ ID NO: 21 | LWT EXPRESSED ON |
| L-BASED VEL-1 LIBRARY | L1 | RECOMBINANT M13 PHAGE IN |
| LVEL1 | 5′GTA TAT TAC TGT GCG NNK | THE CONTEXT OF FLANKING 5AA |
| GVYYGA RGPGXAXXXX | GGT NNK GGT NNK GCT NNK | FROM FR3 Y FR4 AND FUSED |
| WGQGT | GTT NNK ATT TGG GGC CAG | WITH PHAGE CPVIII. |
| | GGA ACC 3′ | VHEXPRESS |
| | SEQ ID NO: 22 | LVEL1 AND LVEL2 EXPRESSED |
| LVEL2 | L2 | IN THE CONTEXT OF MODIFIED V_{H} |
| GVYYGA XGXGXAXGXI | 5′GTA TAT TAC TGT GCG CGT | PG8SAET |
| WGQGT | GGT CCT GGT NNK GCT NNK | LVEL1 AND LVEL2 EXPRESSED |
| | NNK NNK NNK TGG GGC CAG | ON RECOMBINANT M13 PHAGE IN |
| | GGA ACC 3′ | THE CONTEXT OF FLANKING 5AA |
| | SEQ ID NO: 23 | FROM FR3 AND FR4 AND, FUSED |
| PRIMERS FOR PCR | 5DAMP | WITH PHAGE CPVIII. |
| AMPLIFICATION | 5′TGATATTCGTACTCGAGCCAT | |
| SYNTETIC PEPTIDE L | GGTGTATATTACTGTGCG 3′ | |
| RGPGRAFVTI | SEQ ID NO: 24 | |
| SYNTETIC PEPTIDE LIBRARY | 3DAMP | |
| SLVEL1 | 5′ATGATTGACAAAGCTTGGATC | |
| RGPGXAXXXX | CCTAGGTTCCCTGGCCCCA 3′ | |
| SLVEL2 | SEQ ID NO: 25 | |
| XGXGXAXGXI | 5NAMP AND 3NAMP | |
| WILD-TYPE GAG EPITOPE | GPN | VHEXPRESS |
| GPWT | 5′GTA TAT TAC TGT GCG CAG | GPWT EXPRESSED IN THE |
| GVYYGA QA AMQMLKETI | GCT GCT ATG CAG ATG CTT | CONTEXT OF CDR3 OF V_{H}. |
| NE WGQGT | AAG GAG ACT ATT AAC GAG | PG8SAET |
| | TGG GGC CAG GGA ACC 3′ | GPWT EXPRESSED ON |
| | SEQ ID NO: 26 | RECOMBINANT M13 PHAGE IN |
| GAG-BASED VEL-1 LIBRARY | GP1 | THE CONTEXT OF FLANKING 5AA |
| GPVEL1 | 5′GTA TAT TAC TGT GCG CAG | FROM FR3 AND FR4 AND, FUSED |
| GVYYGA QA | GCT GCT ATG NNK ATG CTT | WITH PHAGE CPVIII. |
| AMXMLXXX NE | NNK NNK NNK ATT AAC GAG | VHEXPRESS |
| WGQGT | TGG GGC CAG GGA ACC 3′ | GPVEL1 AND GPVEL2 |
| | SEQ ID NO: 27 | EXPRESSED IN THE CONTEXT OF |
| GPVEL2 | GP2 | MODIFIED V_{H} |
| GVYYGA QA | 5′GTA TAT TAC TGT GCG CAG | PG8SAET |
| AXMXMXETX NE | GCTGCT NNK CAG NNK CTT | GPVEL1 AND GPVEL2 |
| WGQGT | NNK GAG ACT NNK AAC GAG | EXPRESSED ON RECOMBINANT |
| | TGG GGC CAG GGA ACC 3′ | M13 PHAGE IN THE CONTEXT |
| | SEQ ID NO: 28 | OF FLANKING 5AA FROM FR3 |
| PRIMERS FOR PCR | PRIMERS 5NAMP AND 3NAMP OR | AND FR4 AND, FUSED WITH |
| AMPLIFICATION | 5DAMP AND 3DAMP. | PHAGE CPVIII. |
| SYNTETIC PEPTIDE GP | | |
| AMQMLKETI | | |
| SYNTETIC PEPTIDE LIBRARY | | |
| SGPVEL1 | | |
| AMXMLXXX | | |
| SGPVEL2 | | |
| AXMXMXETX | | |

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition comprising a mixture of synthetic peptides, the peptides comprising at least one epitope of a pathogen-specific polypeptide, wherein at least one amino acid residue of the peptides is substituted with each of the other nineteen common amino acid residues in individual peptides of the mixture.

2. The composition of claim 1, wherein every even amino acid residue of the peptides is substituted with each of the other nineteen common amino acid residues.

3. The composition of claim 1, wherein every odd amino acid residue of the peptides is substituted with each of the other nineteen common amino acid residues.

4. The composition of claim 1, wherein the peptides are prepared by chemical synthesis.

5. The composition of claim 1, wherein the peptides are prepared by expression from a nucleic acid construct.

6. The composition of claim 5, wherein the peptides are prepared by expression in a bacterial, viral or eukaryotic expression system.

7. The composition of claim 6, wherein the peptides are expressed and displayed on the surface of a recombinant bacteriophage, bacterium or yeast cell.

8. The composition of claim 1, wherein the epitope of a pathogen-specific polypeptide is selected from the group consisting of one or more epitopes of a Human Immunodeficiency Virus (HIV)-specific polypeptide, a Simian Immunodeficiency Virus (SIV)-specific polypeptide, a Hepatitis A-specific polypeptide, a Hepatitis B-specific polypeptide, a Hepatitis C-specific polypeptide, a rhinovirus-specific polypeptide, an influenza virus-specific polypeptide, and a plasmodium falciparum-specific polypeptide.

9. The composition of claim 24, wherein the epitope of a disease-specific polypeptide is one or more epitopes of a tumor specific or a tumor associated antigen (TAA).

10. A method comprising:
a) preparing a variable epitope library (VEL);
b) injecting the library into a subject; and
c) inducing an immune response in the subject against the VEL.

11. The method of claim 10, wherein preparing a VEL comprises preparing VEL bearing epitopes of a pathogen-specific polypeptide.

12. The method of claim 10, wherein preparing a VEL comprises preparing VEL bearing epitopes of a disease-specific polypeptide.

13. The method of claim 10, wherein inducing the immune response comprises inducing the immune response effective to protect the subject against infection with a pathogen.

14. The method of claim 10, wherein inducing the immune response comprises inducing the immune response effective to treat a subject infected with a pathogen.

15. The method of claim 10, wherein inducing the immune response comprises inducing the immune response effective to protect the subject against a disease.

16. The method of claim 15, wherein the disease is cancer.

17. A composition comprising a mixture of synthetic peptides, the peptides comprising at least one epitope of an human immune deficiency virus (HIV)-specific polypeptide, wherein at least one amino acid residue of the peptides is substituted with each of the other nineteen common amino acid residues in individual peptides of the mixture.

18. The composition of claim 17, wherein either every even numbered amino acid residue or odd numbered amino acid residue of the peptides are substituted with each of the other nineteen common amino acid residues.

19. The composition of claim 17, wherein at least one epitope of HIV-specific polypeptide is at least one epitope of an env-derived CTL epitope.

20. The composition of claim 17, wherein at least one epitope of HIV-specific polypeptide is at least one epitope of a gag-derived CTL epitope.

21. A method comprising:
a) preparing a VEL comprising HIV gag- and env-derived CTL epitopes;
b) injecting the HIV library into a subject; and
c) inducing an immune response in the subject against the HIV VEL.

22. The method of claim 21, wherein inducing an immune response comprises inducing an immune response effective to protect the subject against HIV infection.

23. The method of claim 21, wherein inducing an immune response comprises inducing an immune response effective to treat a subject infected with HIV.

24. A composition comprising a mixture of synthetic peptides, the peptides comprising at least one epitope of a pathogen-specific polypeptide, wherein at least one amino acid residue of the peptides is substituted with each of the other nineteen common amino acid residues in individual peptides of the mixture.

25. The composition of claim 1, wherein the epitope of a pathogen-specific polypeptide is an epitope of a viral pathogen-specific polypeptide.

26. The composition of claim 1, wherein the epitope of a pathogen-specific polypeptide is an epitope of a bacterial pathogen-specific polypeptide.

27. The composition of claim 1, wherein the epitope of a pathogen-specific polypeptide is an epitope of a parasitic pathogen-specific polypeptide.
